(19) United States
(12) Patent Application Publication
(10) Pub. No.: US 2022/0203091 A1
(43) Pub. Date: Jun. 30, 2022

(54) WEARABLE ELECTRODE ARRANGEMENT

(71) Applicant: Brainpatch Limited, London (GB)

(72) Inventor: Nickolai Vysokov, London (GB)

(21) Appl. No.: 17/563,621

(22) Filed: Dec. 28, 2021

(30) Foreign Application Priority Data
Dec. 31, 2020 (GB) 2020827.8

(51) Int. Cl. A61N 1/36; A61N 1/04

(52) U.S. Cl. CPC A61N 1/36025; A61N 1/0476; A61N 1/0456

(57) ABSTRACT

There is disclosed a wearable device that receives electrical signals from a user, wherein the wearable device is in direct contact with skin of the user, the wearable device comprising a plurality of electrodes that, when in operation, makes electrical contact with skin of the user to detect electrical signals therefrom. The wearable device comprises a mechanism that scales the wearable device with shape of a point of contact on which the wearable device is worn for appropriate positioning of the plurality of electrodes.

### TECHNICAL FIELD

The present disclosure relates to wearable devices that receive and deliver electrical signals from a user. Particularly, the present disclosure relates to wearable devices including electrodes which are in direct contact with skin of the user.

### BACKGROUND

Recently, equipment that is operable to stimulate human nervous systems has significantly evolved. Moreover, Non-Invasive Brain Stimulation (NIBS) systems are being contemporarily used for stimulation of brains. In an example, a function of a nervous system is modified by applying electrical stimulation to the nervous system to control a perception of pain by the nervous system, or a different brain stimulation protocol is used to enhance performance of the nervous system when performing cognitive tasks. This function requires tight and low impedance contact of at least one pair of electrodes to the skin. For some applications it is imperative that the electrodes are located on particular spots on the skins and remain there during the course of the stimulation.

Typically, electrochemical signals originating in the given brain of the given person are spread spatially by the skull and can be detected by connecting electrodes in contact with a scalp region of the given person. Generally, signals picked up from the given brain, by using electrodes connected in contact with the scalp region, have an amplitude in an order of tens to hundreds of microvolts. These signals or parameters derived from these signals are linked to various brain states, cognitive activity and particular disorders. It is also possible to use these same or different electrodes to deliver electrical currents for Non-Invasive Brain Stimulation (NIBS). However, the conventional Non-Invasive Brain Stimulation (NIBS) systems requires an expert for fixing the electrodes on the scalp region of the person. Moreover, the conventional design of the electrodes pose difficulty in fixing the electrodes without an expert. Conventionally used brain interfacing devices (BID) and Non-Invasive Brain Stimulation (NIBS) systems required multiple electrodes to be placed on scalp of the person for recording and delivering the brain stimuli. Specifically, some require expert involvement for application of gels, while dry electrodes were often poor quality and are not suitable for stimulation.

Arguably, one of the most complex steps in positioning the electrodes is figuring out their location in a standard 10/20 system. This relies on the distance between naseon and ineon and scales all the locations in proportion to these points. It is worth noting that the distance between the naseon and ineon is different for different individuals. Furthermore, positioning one or more electrodes on the scalp of a number of individuals can be quite troublesome as the actual distance of the electrode to a single fixed arbitrary point is different for each individual. Thus, even if the location of electrodes to be placed on head is fixed in proportions, the distance in mm between the electrodes can vary dramatically.

Conventionally used brain stimulation systems find it really difficult for the electrodes to be placed on the scalp perfectly. Some of the manufacturers of the conventional brain stimulation systems manufacture the brain stimulation devices with one or more size ranges. However, it is not possible to fulfil the size requirements of each and every individual. Furthermore, conventionally used systems roughly fits the head shape of the user and consequently, the process of brain stimulation gets hindered.

Moreover, in the conventional Brain recording systems, accurate determination of electrical signals generated from the brain of the user is difficult. These conventional systems do not provide a good electrical contact, specifically, the systems do not provide the impedance required for good electrical contact for stimulation at right spot on the skin and hair of the user.

Despite advancements that have been made in the aforementioned BID and NIBS, there exists a need to redesign the equipment and electrodes to eliminate the requirement for assistance from an expert for using the device. Furthermore, there is a need to maintain good firm electrical contact at the skin/hair of the user for accurate determination of electrical signals.

### SUMMARY

The present disclosure seeks to provide an easy-to-use wearable electrode arrangement, for example a non-invasive brain stimulation device, that is easy to apply by a user of the device.

In an aspect, the present disclosure provides a wearable device that, when in operation, receives electrical signals from a user, wherein the wearable device is in direct contact with skin of the user, the wearable device comprising:
a plurality of electrodes that, when in operation, makes electrical contact with skin of the user to detect electrical signals therefrom;
a mechanism that scales the wearable device with shape of a point of contact on which the wearable device is worn for appropriate positioning of the plurality of electrodes.

Embodiments of the disclosure are advantageous in terms of providing a wearable electrode arrangement, which is easy to operate and does not require an expert or specialist for positioning the electrodes. Also, the wearable electrode arrangement as disclosed herein, do not use any conductive gel or glue. Furthermore, the devices of the present disclosure provide a solution for achieving safe and effective transcranial stimulation, non-invasive recording of brain activity and real-time optimisation of brain stimuli in accordance with the response received from the brain. Moreover, the devices of the present disclosure provide comfort to the user and low impedance electrical contact for stimulation at right spot on the skin of the user. In other words, the device maintains good electrical contact at the skin of the user at conventionally appropriate points to accurately determine the electrical signals generated by the brain of the user.

Beneficially, the present disclosure provides a wearable brain interfacing device which does not require an expert to assist with efficiently fixing the electrodes to the user's skin for receiving and delivering stimulation to brain of the user.

Optionally, the wearable device includes at least one of a head wearable, a headset, a wristband, an armband and gloves.

Optionally, each electrode of the plurality of electrodes comprises a flexible cushioning member with a conductive medium in an individual housing, wherein the conducting medium includes liquids with an ionic composition to establish an electrical path to detect electrical signals generated by brain of the user.

Optionally, the plurality of electrodes is resiliently flexible for adapting shape of a point of contact.

Optionally, the plurality of electrodes covers an area on the skin of the user in a range of 500-2500 mm².

Optionally, the flexible cushioning member includes at least one of a silicon pad and foam pad for adapting shape of the point of contact of the plurality of electrodes on the skin of the user.

Optionally, the housing includes at least one spring material for applying pressure on the flexible cushioning member and the plurality of electrodes, for embracing the skin of the user.

Optionally, contact between the plurality of electrodes and the skin of the user is conductive gel or glue free.

Optionally, the plurality of electrodes, when in operation, delivers stimulation to brain of the user.

Optionally, the stimulation includes at least one of electrical stimuli, visual stimuli and audio stimuli.

Optionally, the wearable device further comprises a data processing arrangement that, when in operation, processes the detected electrical signals and generates the brain stimuli corresponding to the detected electrical signals.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary embodiments of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a wearable device, in accordance with an embodiment of the present disclosure;

FIGS. 2A and 2B are a schematic illustration of a wearable device, in accordance with an embodiment of the present disclosure;

FIG. 3 is a schematic illustration of a wearable device, in accordance with an embodiment of the present disclosure;

FIG. 4 is a schematic illustration of a wearable device, in accordance with another embodiment of the present disclosure; and

FIG. 5 is a schematic illustration of a wearable device, in accordance with yet another embodiment of the present disclosure.

FIG. 6a is a schematic illustration of a wearable device, when not in use, in accordance with an embodiment of the present disclosure.

FIG. 6b is a schematic illustration of the wearable device provided in FIG. 6a, when in use, in accordance with an embodiment of the present disclosure.

FIG. 7a is a schematic illustration of another wearable device, in a non-extendable position, in accordance with an embodiment of the present disclosure.

FIG. 7b is a schematic illustration of the wearable device as provided in FIG. 7a, in an extendable position, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item to which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognise that other embodiments for carrying out or practising the present disclosure are also possible.

In an aspect, the present disclosure provides a wearable device that, when in operation, receives electrical signals from a user, wherein the wearable device is in direct contact with skin of the user, the wearable device comprising:
a plurality of electrodes that, when in operation, makes electrical contact with skin of the user to detect electrical signals therefrom;
a mechanism that scales the wearable device with shape of a point of contact on which the wearable device is worn for appropriate positioning of the plurality of electrodes.

Throughout the present disclosure, the term “user” as used herein relates to any person (i.e., human being) using the aforesaid device. Optionally, the user may be a person having a certain physical or mental disorder such as epilepsy, a head injury, encephalitis, brain tumour, encephalopathy, memory related problems, sleep disorders, stroke, dementia, depression, etc. Alternatively, the user may be a person willing to achieve a specific state of mind, such as an enhanced concentration, relaxation, mental capabilities or, in general terms, enhanced performance for executing a task.

Throughout the present disclosure, the term “brain activity monitoring” as used herein relates to detection of electrical signals received from the brain by a method of electroencephalography (EEG). Optionally the brain activity monitoring includes detection of signals which include, but are not limited to, signals, or a combination of signals, obtained using Electromyography (EMG), Electrocardiography (ECG) or Galvanic Skin Response (GSR) including signals coming from electrodes located spatially remote from the given user's scalp. More optionally, the brain activity monitoring relates to monitoring of a change in electrical activity of the brain of a user, upon providing external electrical stimulus to the brain of the user. More optionally, the electrical activity of the brain of a user may be indicative of biological parameters related to the mental and physical health of a user including, but not limited to, a heart rate, a breathing rate and a skin conductance.

Throughout the present disclosure, the term “electrode”, “at least one electrode”, “plurality of electrodes” or “elongated electrode” as used herein relates to one or more electrical conductors, with the materials of these conductors including, but not limited to, stainless steel, platinum, sliver chloride-coated silver, carbon rubber, graphene and other metamaterials, as well as hydrogels, silicone, sponges, foam or any absorbent with a conducting medium, where necessary to be placed between the conductors and the scalp or skin. The conducting medium may include liquids (such as physiological saline solution) with such an ionic composition as to establish an electrical path to detect electrical signals generated by the neurons inside the brain and to provide brain stimuli to the neurons and/or other cells present inside the brain of the user. Furthermore, the electrodes are operable to convert an ionic potential into an electric potential and to induce electromagnetic fields on the scalp and inside the skull. Moreover, the electrodes can be of minimally invasive (such as needle electrodes or micro electrodes) or non-invasive type (such as surface electrodes), or optionally both. In an example, the electrodes comprise an assembly of saline-soaked foam, conductive carbon and a metal contact. In such an example, the metal contact is operatively coupled with one or more components of the brain interfacing device (such as, an input/output arrangement and/or a data processing arrangement, described in detail herein later).

The flexible cushioning member with the conducting medium acts as an electrode, wherein the flexible cushioning member is in direct contact with electrically conductive part of the brain stimulation system and at the other end, it is in direct contact with the skin/hair of the user. The conducting medium is in contact with the skin of user either directly or through hairs of the user. This arrangement provides comfort to the user, moreover, it results into low impedance electrical contact for stimulation at right spot on the skin of the user.

The wearable device pursuant to the present disclosure, when in operation, receives electrical signals from a user. Specifically, the wearable device is in direct contact with skin of the user. The wearable device comprises a plurality of electrodes that, when in operation, makes electrical contact with the skin of the user to detect electrical signals therefrom. The wearable device further comprises the plurality of electrodes being positioned on the skin of the user such that the at least one electrode embraces the skin of the user, wherein each electrode comprises a flexible cushioning with a conductive medium in an individual housing. Further, the wearable device includes a housing for accommodating the flexible cushioning member and the plurality of electrodes therein, and positioning the wearable device on the skin of the user, at an appropriate position.

Throughout the present disclosure, the term “appropriate position” as used herein relates to a suitable position of the plurality of electrodes on skin of the user for accurate determination of the electrical signal at that particular point of contact or a derivative of the signal from a plurality of such suitable positions. In the context of NIBS or closed-loop stimulation, the same term relates to a suitable position of the plurality of electrodes on the skin of the user for stimulation of a brain area located under the electrode or to a combination of suitable positions of electrodes giving rise to focused or network-specific neurostimulation.

As previously discussed, the electrodes to be placed on the scalp of an individual are required to comply with the 10/20 electrode placement system. The 10/20 system is based on the relationship between the location of an electrode and the underlying area of cerebral cortex. The numbers “10” and “20” refer to the fact that the distances between adjacent electrodes are either 10% or 20% of the total front-back or right-left distance of the skull.

Throughout the present disclosure, the term “Cz position” or “Cz region” as described herein relates to a specific reference position at which the electrodes have to be placed in accordance with the 10/20 electrode positioning convention. The “Cz position” relates to the point at which the length between the naseon (or ineon) and Cz position is the distance of fifty percent of the total front back or right left distance of the skull. In other words, the “Cz position” relates to a midline central point of a user's skull.

Throughout the present disclosure, the term “T3 position or region” as described herein relates to a specific reference point at which the electrodes are to be placed. The “T3 position” is the reference points at 40% of the distance between the nasion and inion laterally towards the left ear. The other reference point “T4” relates to its counterpart at the opposite side of the T3 position.

Other positions used or implied in this embodiment, such as “C3 position” or “C4 position” (as well as “Fp1”, “Fpz”, “Fp2”, “F7”, “F6”, “T5”, “T6”, O“1”, “Oz”, “O2” etc.) are derived in a similar manner in relation to the nasion, inion and the Cz position in accordance with the 10/20 convention.“M1” and “M2” positions refer to the left and right mastoid regions behind the ear respectively.

In one of the embodiments of the present disclosure, a wearable device is disclosed that is implemented in the form of a headband placed or positioned around the forehead of a user. The headband, when in operation, receives and delivers stimulations to and from the brain of an individual. The wearable device implemented in the form of headband comprises a plurality of electrodes, wherein each electrode comprises a flexible cushioning with a conductive medium in an individual housing. The plurality of electrodes of the wearable device makes electrical contact with skin of the user to detect electrical signals therefrom and to apply brain stimuli thereto. The flexible cushioning member flexes in response to the plurality of electrodes being positioned over the head of the user such that the plurality of electrodes embraces the skin of the user. Specifically, the headband comprises a mechanism that scales the headband with shape of a point of contact on which the headband is worn for appropriate positioning of the plurality of electrodes. In particular, headband comprises a stretchable unit that stretches out equally in all directions. Therefore, the one or more electrodes are appropriately positioned on the scalp of the user, as in accordance with the requirements of 10/20 electrode placement system. In this embodiment, the stretchable unit automatically stretches out equally in all directions as the user tries to put the headband on. Consequently, the one or more electrodes are appropriately positioned over the mastoid regions (M1 and M2) of the user's brain to facilitate the wearable device for receiving electric signals from the user's brain and to applying brain stimuli thereto. The mastoid region as described herein is a part of the user's skull that is located just behind the user's brain. In this embodiment, from the plurality of electrodes, a front electrode is placed in Fp1 (left side), FpZ (centre) Fp2 (right-side) and side electrodes end up in T3(left)/T4(right) and the back electrodes end up in O1 (left) and O2 (right). Furthermore, in this embodiment, the one or more electrodes are appropriately positioned at the Fp1, Fpz, Fp2, T3, T4, O1, O2 as well as at the mastoid regions (either M1 or M2 or both) of the user's brain. The plurality of electrodes of the wearable device makes electrical contact with skin of the user to detect electrical signals therefrom and to apply electrical stimuli thereto.

In another embodiment, the headband further comprises an accelerometer for detecting the correct orientation so as to facilitate the appropriate positioning of the plurality of electrodes in relation to the nasion and the inion of the user. In yet another embodiment, the headband further comprises a stretchable unit that is appropriately stretched so as to position the one or more electrodes at the Fp1, FpZ, Fp2, F7, F6, T5, T6, T3, T4, T5, T6, O1, Oz and O2 positions of the scalp of the user.

The wearable device of the present invention is advantageous in terms of portability and user friendliness. The wearable device of the present invention is susceptible to working robustly on different head sizes, namely small heads as well as large heads. The wearable device of the present invention may be used as a home held brain interfacing device, for example in a manner as the user could wear the headphones without any help from an expert. Thus, the wearable device of the present invention eliminates the requirement of an expert to assist for fixing the electrodes.

In an embodiment, the wearable device may be implemented in form of at least one of a head wearable, a headset, a wristband, an armband and gloves. Further, the head wearable may include but not limited to a head band, a cap, a hat, a pair of glasses or any other head wearable.

In another embodiment, the plurality of electrodes is resiliently flexible for adapting shape of a point of contact. Advantageously, resiliently flexible nature of the plurality of electrodes facilitates adaptation of the shape of the appropriated position on the skin of the user.

In another embodiment, the plurality of electrodes covers an area on the skin of the user in a range of 500-2500 mm². For example, the area covered by the plurality of electrodes on the skin of the user may be 500, 510, 520, 530, 540 . . . and so forth.

In an embodiment, the flexible cushioning member may include at least one of a silicon pad and foam pad for adapting shape of the point of contact of the plurality of electrodes on the skin of the user. Specifically, the flexible cushioning member adapts the shape of a surrounding region of the appropriate position of the plurality of electrodes.

In another embodiment, the housing may include at least one spring material for applying pressure on the flexible cushioning member and the at least one electrode, for embracing the skin of the user.

In another embodiment, contact between the at least one electrode and the skin of the user is conductive gel or glue free. Beneficially, the conductive gel or glue free contact between the at least one electrode and the skin of the user eases the process of setting up the electrodes to a point where no expert involvement is strictly necessary, and reduces the discomfort associated with having gel or glue on skin/hair.

In another embodiment, the at least one electrodes, when in operation, may deliver stimulation to brain of the user. Further, the stimulation may include at least one of, but not limited to electrical, visual and audio stimuli.

Throughout the present disclosure, the terms “brain stimulus”, “brain stimuli” (plural of “stimulus”) or “stimulation” as used herein relates to an external electrical current or to a defined sequence or multiple sequences of electric current amplitudes between a pair, several pairs or any combination of the electrodes applied to the scalp of a user or to locations spatially remote from the scalp of a user, in order to alter (referring to raising, lowering or otherwise modulating) levels of physiological or nervous activity in the brain or in the tissues spatially remote from the given user's brain that the current is able to reach. Moreover, in an example, brain stimuli applied to the scalp of the user are analogue external electrical signals having a voltage in a range of 1 millivolt to 50 volts and having a current in a range of 0.1 milliampere to 20 milliamperes.

In another embodiment, the wearable device may include a data processing arrangement that, when in operation, processes the detected electrical signals and generates the brain stimuli corresponding to the detected electrical signals.

Throughout the present disclosure, the term “data processing arrangement” as used herein relates to programmable and/or non-programmable components that, when in operation, execute one or more software applications for storing, processing and/or sharing of data and/or a set of instructions. Optionally, the data processing unit can include, for example, a component included within an electronic communications network. Furthermore, the data processing arrangement may include hardware, software, firmware or a combination of these, suitable for storing and processing various information and services accessed by the one or more user using the one or more user equipment. Optionally, the data processing arrangement may include functional components, for example, a processor, a memory, a network adapter and so forth. For example, the data processing arrangement can be implemented using a computer, a phone (for example, a smartphone), a local server, a server arrangement (such as, an arrangement of two or more servers communicably coupled with each other), a cloud server, a quantum computer and so forth.

Furthermore, the electrode, when actively delivering current and when in contact with the skin of the user, applies electromagnetic fields to the brain of the user acting as brain stimuli. Such brain stimuli are provided with the help of generated brain stimulation protocols received by an input/output arrangement from the data processing arrangement. Throughout the present disclosure, the term “input/output arrangement” as used herein relates to programmable and/or non-programmable components that, when in operation, receive, modify, convert, process or generate one or more types of signals. Optionally, the input/output arrangement is implemented as a hardware or a software, or a combination thereof. The generated brain stimulation protocols received from the data processing arrangement are processed by the input/output arrangement, namely converted, into an analogue form and adjusted to a desired current amplitude, before being applied as brain stimuli to the scalp of the user.

Whenever the stimuli are applied like this in a recursive manner, it is referred to herein as a “closed-loop” stimulation.

In an embodiment, a comparison of processed electrical signals or a set of parameters extracted from the signals with the predetermined reference data set is performed, for example, with the help of a comparator or one or more artificial intelligence algorithms or other data processing algorithms implemented in the processing unit of the data processing arrangement. Thereafter, the data processing arrangement generates an analysis of the compared electrical signals. Furthermore, the analysis optionally includes a measure of at least one: of a deviation of a parameter derived from an ideal reference signal stored in the predetermined reference data set; of a reason for such deviation from the ideal reference signal; and/or of a parameter derived following decomposition of the waveforms by individual component analysis, principal component analysis or Fourier transformation, periodogram, wavelet decomposition, wavelet transform, adaptive filters such as Wiener/Kalman filters, and other methods commonly used by those skilled in the art.

The werable device comprises a mechanism that scales the wearable device with shape of a point of contact on which the wearable device is worn for appropriate positioning of the plurality of electrodes. In a specific embodiment, the wearable device is implemented as a headset. Specifically in this embodiment, the plurality of electrodes is placed or positioned over the ear of the user, specifically the at least one electrode is in direct contact with skin behind the ear, such as on the mastoid (M1 and M2) regions, in order to establish an electrical contact with the skin and subsequently with the neurons in the brain of the user. Such an electrical contact establishes an electrical path between these electrodes or in conjunction with the other electrodes to detect electrical signals generated by the neurons and to provide brain stimuli to the neurons and/or other cells present inside the brain of the user. A pair of electrodes is able to detect the electromagnetic signals generated inside the brain of the user by activity of neurons, wherein the detected electrical signals are provided to the input/output arrangement. Generally, the amplitude of the electrical signals detected on the scalp of the user ranges between 1 microvolt to 100 microvolts. The electrode may optionally be configured as any suitable EEG electrode arrangement known in the art and the headset adjustment aligns the electrode at an appropriate position.

. In an embodiment, the wearable device is implemented as a headset that comprises one or more extendable units positioned between the top and the middle side of the headset and a plurality of electrodes, wherein each electrode comprises a flexible cushioning with a conductive medium in an individual housing. The plurality of the electrodes of the wearable device makes electrical contacts with skin of the user to detect electrical signals therefrom and to apply brain stimuli thereto. Specifically, in this embodiment, with the extension of the extendable units that automatically takes place as the user tries to put the headset on, the plurality of electrodes are positioned appropriately at the side positions (C3 and C4 position) of the scalp of the user along with the appropriately positioning of plurality of electrodes at the temple(s) regions of the brain (T3 and T4).

Furthermore, in another embodiment, the headset as described herein comprises a flexible and stretchable unit positioned at the top side of the headset. The flexible and stretchable unit positioned at the top side of the headset, facilitates in positioning of the plurality of electrodes appropriately at the side regions (C3 and C4) as well as temporal regions (T3 and T4) of the scalp of the user by way of stretching equally in all directions in a manner similar to the headband described above. In another embodiment of the present disclosure, the headset further comprises an accelerometer to detect the correct orientation of the sagittal plane of the scalp of a user and thus, facilitates in positioning of one or more electrodes at the Cz region of the scalp of the user. In another embodiment, the wearable device is implemented in the form of a clip placed or positioned on ear(s) of the user. Specifically, in this embodiment, the wearable device includes a plurality of electrodes, wherein each electrode comprises a flexible cushioning with a conductive medium in an individual housing. The plurality of electrodes of the wearable device makes electrical contact with skin of the user to detect electrical signals therefrom and to apply brain stimuli thereto between the electrodes or conjunction with electrodes positioned elsewhere in accordance with the other embodiments. The flexible cushioning member of wearable device supports the plurality of electrodes, and flexes in response to the plurality of electrodes being positioned over the ear(s) of the user such that the plurality of electrodes embraces the ear(s) of the user and is aligned at an appropriate position.

In an additional embodiment, the wearable device is implemented as a wrist wearable device that, when in operation, receives and delivers stimulation to brain of a user. The wearable device includes a plurality of electrodes, wherein each electrode comprises a flexible cushioning with a conductive medium in an individual housing. The plurality of electrodes of the wearable device makes electrical contact with skin of the user to detect electrical signals therefrom and to apply brain stimuli thereto. The flexible cushioning members of wearable device flex in response to the electrode being positioned over the wrist of the user such that the plurality of electrodes embraces the wrist of the user and aligns the electrode at an appropriate position.

In a particular embodiment of the present disclosure, the wearable device is implemented in the form of a combination of a headband and other wearable devices as described herein the present disclosure, for the purpose of delivering and receiving electric signals to and from the user's brain. In an example, the wearable device may be implemented in the form of a combination of a headband configuration and a headset configuration. The plurality of electrodes provided by the wearable device, are placed or positioned over the ear of the user, specifically the at least one electrode is in direct contact with skin behind the ear, such as on the mastoid (M1 and M2) regions, in order to establish an electrical contact with the skin and subsequently with the neurons in the brain of the user as in accordance with an embodiment of the present disclosure. Furthermore, as in accordance with other embodiments, the plurality of electrodes of the wearable device can be positioned elsewhere as in accordance with the requirements of 10/20 electrode positioning system as discussed in other embodiments.

In an embodiment, the wearable device is implemented in the form of combination of headband and clip placed on ear(s) or other positions on the scalp of the user, as described in the embodiments of the present disclosure. Alternatively, the wearable device is implemented in the form of combination of headset and clip placed on ear(s) or other position on the scalp of the user. In an another alternative arrangement, the wearable device may also be implemented in the form of a combination of headband and wrist wearable device or the combination of clips placed on the user's scalp/ear and the wrist wearable device as described by the embodiments of the present disclosure. The wearable device as provided herein, is able to deliver and detect electric signals to and from the brain of the user by placing the electrodes on the strategic positions of user's scalp as discussed in the embodiments of the present disclosure.

In an embodiment, the plurality of electrodes are hybrid electrodes which can function as both for EEG recording and/or for electrical stimulation, for example, transcranial current stimulation (tCS), transcranial direct current stimulation (tDCS), transcranial alternating current stimulation (tACS), transcranial random noise stimulation (tRNS), transcranial temporal interference stimulation (TI), transcranial temporal summation (TS) and/or any other arbitrary transcranial electric current stimulation protocol generated by the adaptive algorithms (tES).

Beneficially, the adaptive learning algorithm contributes largely in achieving a more personalised and thus more effective brain stimulation for the user. Additionally, the adaptive learning algorithm or another computational algorithm continuously, in a closed loop manner, learns the patterns of response of the brain of the user to the past stimulation to better adjust the future brain stimuli for achieving optimised results. Furthermore, implementation of adaptive learning algorithms helps in enhancing the therapeutic contribution of neuromodulation devices such as the brain interfacing apparatus of the present disclosure.

Beneficially, the control unit and the third-party devices provide a better interaction with the user through a user-friendly interface. Optionally, the control unit enables the third-party device to execute a customised adaptive learning algorithm instead of the data processing unit, which can be beneficial where the processing power required for the execution of the adaptive learning algorithm exceeds that of a data processing unit.

In a specific embodiment, the wearable device pursuant to the present disclosure, when in operation, receives and delivers stimulation to brain of the user. The wearable device comprises a plurality of electrodes that, when in operation, makes electrical contact with skin of the user to detect electrical signals therefrom and to apply brain stimuli thereto. The wearable device further comprises an additional plurality of electrodes wherein each electrode comprises a flexible cushioning with a conductive medium in an individual elongated housing positioned over the ear(s) of the user such that the plurality of electrodes embraces the ear(s) of the user; “ear(s)” means “one ear or both ears”. The wearable device also includes a data processing arrangement that processes the detected electrical signals and generates the brain stimuli corresponding to the detected electrical signals. Particularly, the at least one electrode is in direct contact with skin on the ear(s) of the user.

### DETAILED DESCRIPTION OF THE DRAWINGS

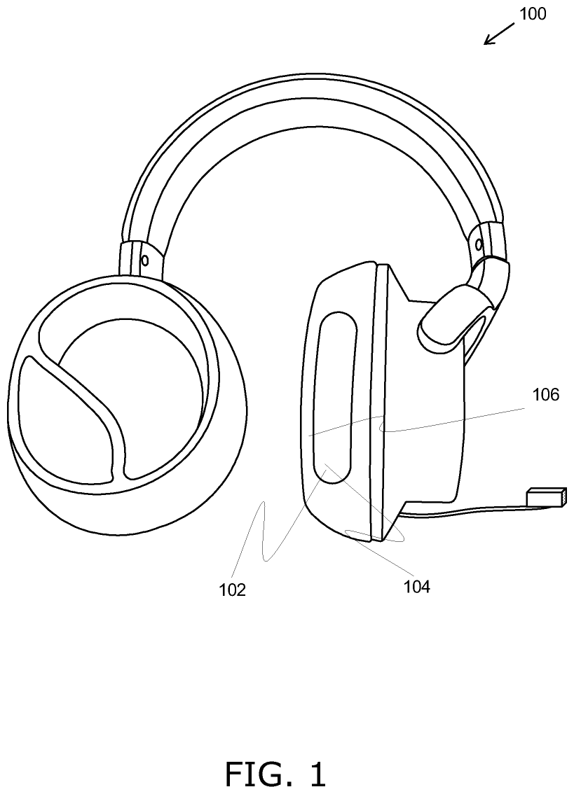

Referring to FIG. 1, there is illustrated a block diagram of a wearable device 100 that, when in operation, receives and delivers stimulation to brain of a user. The wearable device 100 includes a plurality of electrodes 102, wherein each electrode comprises a flexible cushioning member 104 and a housing 106. The plurality of electrodes 102 of the wearable device 100 makes electrical contact with skin of the user to detect electrical signals therefrom and to apply brain stimuli thereto. The flexible cushioning member 104 of wearable device 100 supports the plurality of electrodes 102, and flexes in response to the electrode 102 being positioned over the ear(s) of the user such that the electrode 102 embraces the skin near the ear(s) of the user.

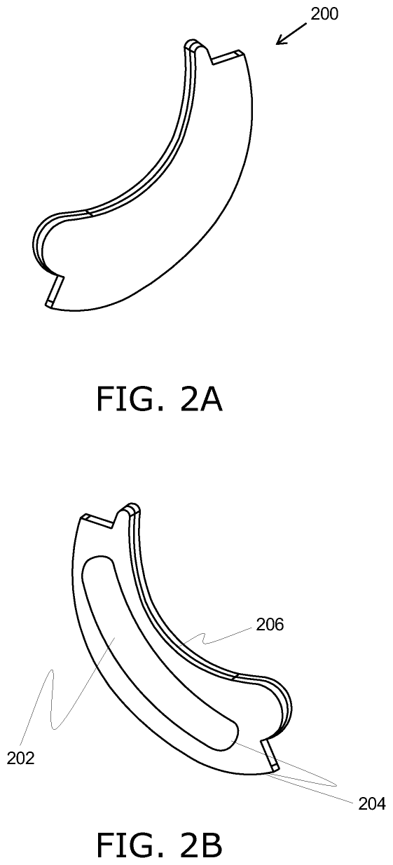

Referring to FIGS. 2A and 2B, there is illustrated a block diagram of housing for an electrode 202 of the plurality of electrodes. The housing 200 includes theelectrode 202, wherein the electrode comprises a flexible cushioning member 204 and a housing 206. The electrode 202 of the housing 200 makes electrical contact with skin of the user to detect electrical signals therefrom and to apply brain stimuli thereto. The flexible cushioning member 204 supports the electrode 202, and flexes in response to the electrode 202 being positioned over the ear(s) of the user such that the electrode 202 embraces the ear(s) of the user.

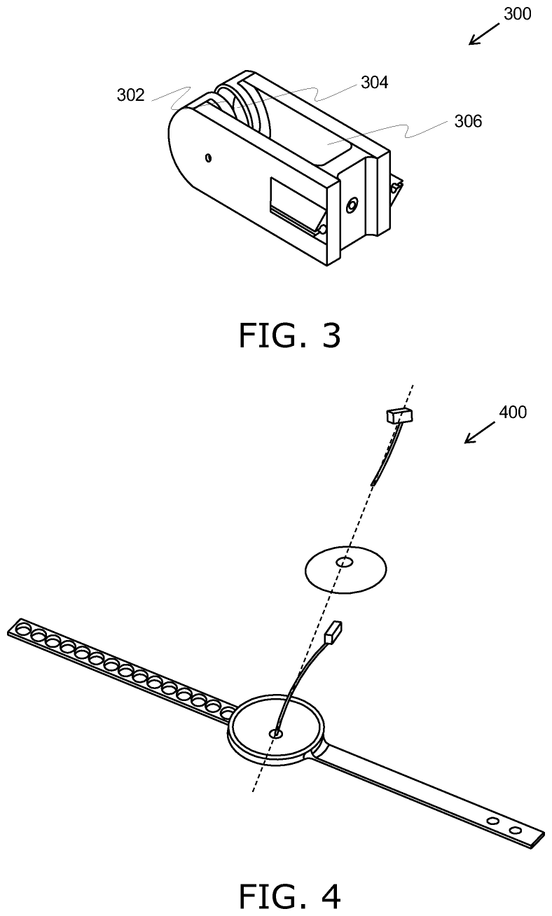

Referring to FIG. 3, there is illustrated a block diagram of another wearable device 300 implemented in the form of a clip that, when in operation, receives and delivers stimulation to brain of a user. The wearable device 300 includes a plurality of electrodes 302, wherein each electrode comprises a flexible cushioning member 304 and a housing 306. The plurality of electrode 302 of the wearable device 300 makes electrical contact with skin of the user to detect electrical signals therefrom and to apply stimuli thereto. The flexible cushioning member 304 of wearable device 300 flexes in response to the plurality of electrodes 302 being positioned over the ear(s) of the user such that the plurality of electrodes 302 embraces a part of the ear(s) of the user.

Referring to FIG. 4, there is illustrated a block diagram of another wearable device 400 implemented in the form of a wrist wearable device that, when in operation, receives and delivers stimulation to brain of a user. The wearable device 400 includes a plurality of electrodes, wherein each electrode comprises a flexible cushioning with a conductive medium in an individual housing. The plurality of electrodes of the wearable device 400 makes electrical contact with skin of the user to detect electrical signals therefrom and to apply brain stimuli thereto. The flexible cushioning member of wearable device 400 flexes in response to the elongate electrode being positioned over the wrist of the user such that the plurality of electrodes embraces the wrist of the user.

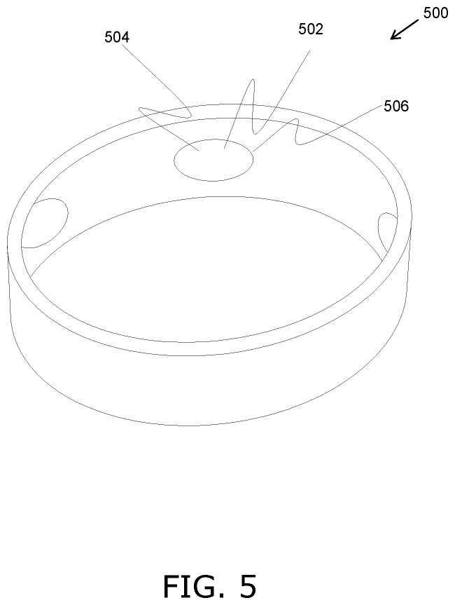

Referring to FIG. 5, there is illustrated a block diagram of another wearable device 500 implemented in the form of a headband that, when in operation, receives and delivers stimulation to brain of a user. The wearable device 500 includes a plurality of electrodes 502, wherein each electrode comprises a flexible cushioning member 504 and a housing 506. The plurality of electrodes 502 are placed or positioned over the ear(s) of the user, specifically the at least one electrode 502 is in direct contact with the scalp in specific regions of the head, such as Fp1, FpZ, Fp2, T3, T4, O1 and O2, or other strategic positions of the user's brain, in order to establish an electrical contact with the skin and subsequently with the neurons in the brain of the user. Such an electrical contact establishes an electrical path between these electrodes or in conjunction with the other electrodes to detect electrical signals generated by the neurons and to provide brain stimuli to the neurons and/or other cells present inside the brain of the user. The flexible cushioning member 504 of wearable device 500 flexes in response to the plurality of electrodes 502 being positioned over temple(s) of the user such that the plurality of electrodes 502 embraces the scalp on the head of the user.

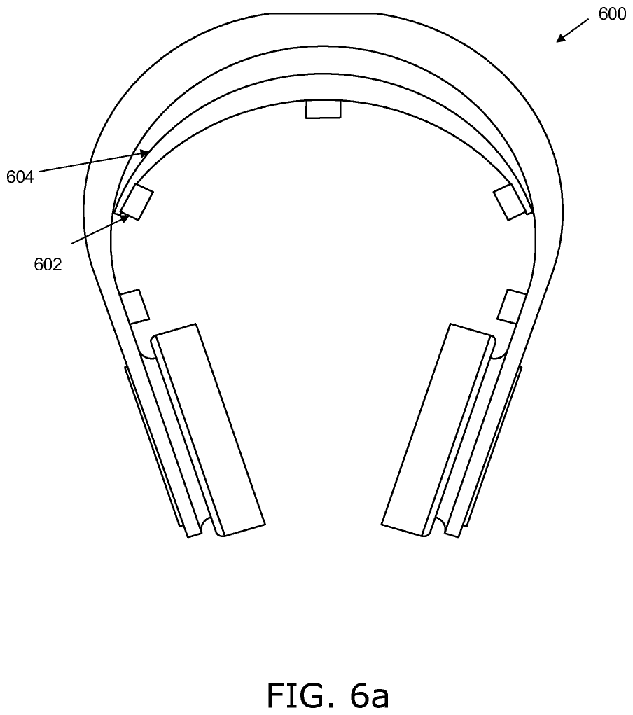

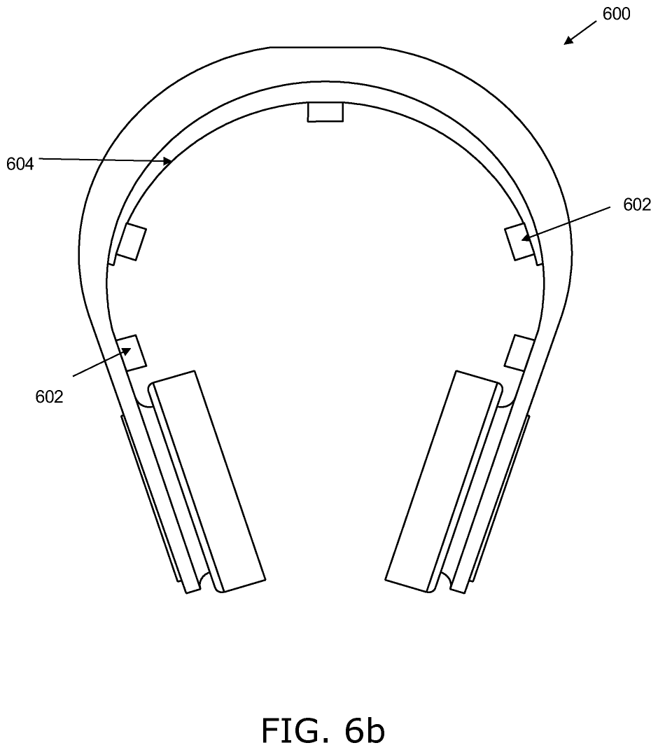

Referring to FIG. 6a and FIG. 6b, there is illustrated the block diagrams of another wearable device 600 implemented in the form of a headset. FIG. 6a is a schematic illustration of a wearable device, when not in use or not in operation and FIG. 6b is another schematic illustration of the wearable device provided in FIG. 6a, when in use or when in operation, in accordance with an embodiment of the present disclosure. FIG. 6b indicates the wearable device 600, implemented in the form of headset that when in operation, receives and delivers signals to brain of a user. The wearable device 600 comprises one or more flexible and stretchable unit 604 positioned at the top side of the headset and a plurality of electrodes 602, wherein each electrode comprises a flexible cushioning with a conductive medium in an individual housing The flexible and stretchable unit 604 is stretched in order to facilitate the positioning of the plurality of electrodes 602 at the side positions (C3 and C4 positions) of the scalp of the user along with the appropriately positioning of plurality of electrodes at the temple(s) regions or positions of the brain. The plurality of electrodes 602 of the wearable device makes electrical contact with skin of the user to detect electrical signals therefrom and to apply brain stimuli thereto.

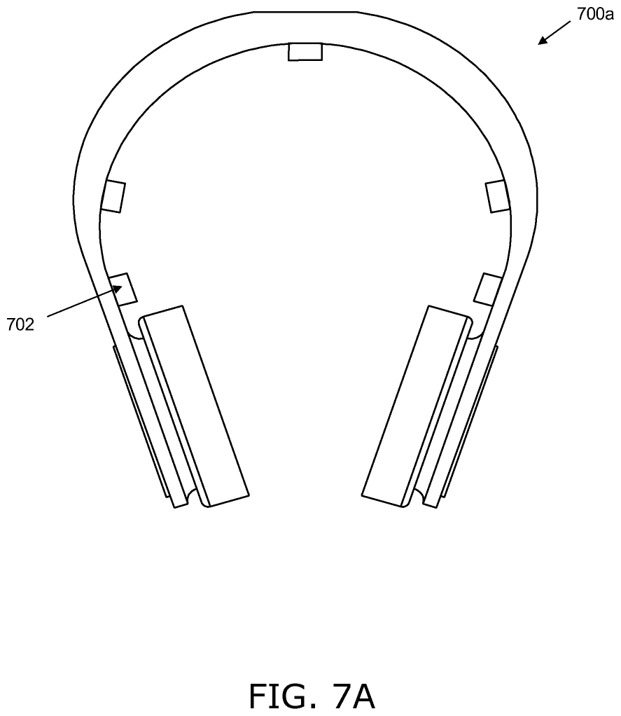

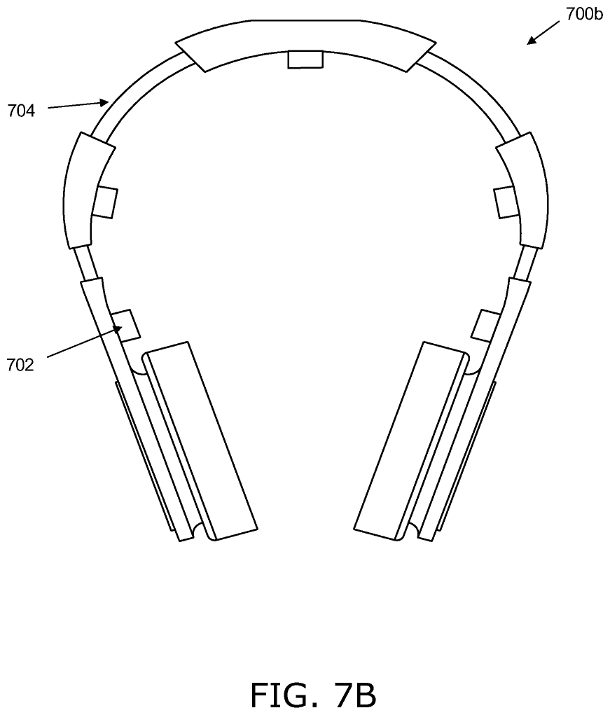

Referring to FIG. 7 of the present disclosure, there is illustrated the block diagram of another wearable device 700a and 700b implemented in the form of a headset. FIG. 7a indicates a wearable device 700a, that is provided in a non-extendable position and FIG. 7b indicates the wearable device 700b, in an extendable position, in accordance with an embodiment of the present disclosure. The wearable device receives and delivers signals to brain of a user and comprises one or more extendable units 704 positioned between the top and the middle side of the headset and a plurality of electrodes 702, wherein each electrode comprises a flexible cushioning member with a conductive medium in an individual housing. Specifically, with the simultaneous extension of the one or more extendable unit 704, the plurality of electrodes are positioned appropriately at the side positions (C3 and C4 position) of the scalp of the user along with the appropriately positioning of plurality of electrodes at the temple(s) regions of the brain. The plurality of electrodes 702 of the wearable device makes electrical contact with skin of the user to detect electrical signals therefrom and to apply brain stimuli thereto.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural where appropriate.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

1. A wearable device that, when in operation, receives electrical signals from a user, wherein the wearable device is in direct contact with skin of the user, the wearable device comprising:
a plurality of electrodes that, when in operation, makes electrical contact with skin of the user to detect electrical signals therefrom;
a mechanism that scales the wearable device with shape of a point of contact on which the wearable device is worn for appropriate positioning of the plurality of electrodes.

2. The wearable device of claim 1, wherein the wearable device includes at least one of a head wearable, a headset, an earclip, an ear cover, a wristband, an armband and gloves.

3. The wearable device of claim 1, wherein each electrode of the plurality of electrodes comprises a flexible cushioning member with a conductive medium in an individual housing, wherein the conducting medium includes liquids with an ionic composition to establish an electrical path to detect electrical signals generated by brain of the user.

4. The wearable device of claim 1, wherein the at plurality of electrodes covers an area on the skin of the user in a range of 500-2500 mm².

5. The wearable device ofclaim 3, wherein the flexible cushioning member includes at least one of a silicon pad and foam pad for adapting shape of the points of contact of the plurality of electrodes on the skin of the user.

6. The wearable device of claim 3, wherein the housing includes at least one spring material for applying pressure on the flexible cushioning member and the plurality of electrodes, for embracing the skin of the user.

7. The wearable device of claim 3, wherein a contact between the plurality of electrodes and the skin of the user is conductive gel or glue free.

8. The wearable device of claim 3, wherein the plurality of electrodes, when in operation, delivers stimulation to brain of the user.

9. The wearable device of claim 8, wherein the stimulation includes at least one of electrical stimuli, visual stimuli and audio stimuli.

10. The wearable device of claim 1, wherein the wearable device further comprises a data processing arrangement that, when in operation, processes the detected electrical signals and generates the brain stimuli corresponding to the detected electrical signals.
